(19) United States
(12) Patent Application Publication
(10) Pub. No.: US 2024/0099960 A1
(43) Pub. Date: Mar. 28, 2024

(54) DENDROBIUM FIMBRIATUM EXTRACT AND NO-RINSE COSMETIC COMPOSITION COMPRISING SAME

(71) Applicant: L V M H RECHERCHE, Saint-Jean-De-Braye (FR)

(72) Inventors: Lorène GOURGUILLON, Saint-Jean-De-Braye (FR); Catherine HEUSEULE, Saint-Jean-De-Braye (FR); Anne-Laure BULTEAU, Saint-Jean-De-Braye (FR); Lauren SOBILO, Saint-Jean-De-Braye (FR)

(73) Assignee: L V M H RECHERCHE, Saint-Jean-De-Braye (FR)

(21) Appl. No.: 18/265,935

(22) PCT Filed: Dec. 13, 2021
(86) PCT No.: PCT/FR2021/052301

§ 371 Date: Jun. 7, 2023

(30) Foreign Application Priority Data
Dec. 18, 2020 (FR) FR2013687

(51) Int. Cl. A61K 8/9794; A61K 8/60; A61K 31/7034; A61K 36/8984; A61Q 19/08

(52) U.S. Cl. CPC A61K 8/9794; A61K 8/602; A61K 31/7034; A61K 36/8984; A61Q 19/08; A61K 2236/15; A61K 2236/333; A61K 2236/39; A61K 2236/53

(57) ABSTRACT

The present invention relates to a no-rinse cosmetic composition for topical application to keratin materials, in particular on the skin and/or the lips and in particular the skin of the face and/or of the neck, comprising an extract of Dendrobium fimbriatum.

### FIELD OF THE INVENTION

The present invention relates to the field of cosmetics. It relates, in particular, to a no-rinse cosmetic composition comprising an extract of orchid from the species Dendrobium fimbriatum, as well as its use for preventing and/or reducing the signs of skin ageing. It also relates to a particular extract of Dendrobium fimbriatum and an extraction method for obtaining such an extract.

### PRIOR ART

The skin is the first barrier for protecting an organism from the environment. Is therefore subject to many factors which can induce signs of skin ageing, whether they be of exogenous origin (e.g. UV radiation, temperature variations, atmospheric pollution, cigarette smoke, etc.) or endogenous origin (e.g. hormones, etc.). These are respectively referred to as extrinsic ageing and intrinsic (or physiological) ageing.

Extrinsic ageing causes clinical changes, such as deep wrinkles and the formation of skin which has lost its firmness, suppleness and elasticity, and the appearance of skin pigment spots, otherwise known as age spots. Concurrently, intrinsic or physiological ageing causes, in particular, a slowing of skin cell renewal and a change in the dermoepidermal junction. The dermoepidermal junction is essential for the anchoring the cells of the epidermis to the underlying tissue, the dermis. The extracellular matrix (ECM) is a complex network composed of an assembly of macromolecules organised in a specific manner, which links them to one another. For the skin, it confers properties of elasticity, strength and compressibility on the one hand, and on the other hand regulates various cellular functions such as the proliferation, migration or differentiation of skin cells. Thus, in the dermis, the ECM plays essential roles in the physiology of the skin. Its alteration will directly impact the quality of the skin, which is essentially manifested by a loss of firmness, elasticity and the appearance of fine wrinkles or fine lines.

In this context, there is a constant need to find novel “anti-ageing” agents which act on biological targets, in particular to prevent and/or reduce the signs of skin ageing.

Unexpectedly, the Applicant has discovered in vitro the effects of an extract of Dendrobium fimbriatum on certain proteins of the dermal ECM. In particular, the extract of Dendrobium fimbriatum according to the invention is capable of significantly increasing the synthesis of elastin, fibrillin 1, collagen 1 and nidogen. In addition, this extract can also reduce in vitro the morphological markers of senescence of fibroblasts. Hence, the extract of Dendrobium fimbriatum according to the invention has a cosmetic interest as an anti-ageing agent, in particular for preventing and/or reducing the signs of ageing such as loss of firmness, loss of elasticity, reduction in thickness of the skin, and the appearance of wrinkles and/or fine lines.

### SUMMARY OF THE INVENTION

The first object of the invention relates to a no-rinse cosmetic composition for topical application to keratin materials, in particular on the skin and/or the lips, and in particular the skin of the face and/or of the neck, comprising an extract of Dendrobium fimbriatum. A “no-rinse cosmetic composition” according to the invention means a care composition intended to remain in contact with keratin materials after application, in order to confirm its beneficial effect (moisturising, anti-ageing, etc.), such as milks, creams, pomades, salves, sticks, gels, lotions, serums, etc., as opposed to “rinsed cosmetic compositions” for cleaning keratin materials (e.g. shower gels, shampoos), intended to be rinsed after application.

The term “keratin materials” according to the invention means the skin and/or its appendages, and the lips. In particular, it involves the skin of the face, the neck and/or the body, and the lips.

The invention also relates to a cosmetic use of an extract of Dendrobium fimbriatum as an agent for preventing and/or reducing the signs of skin ageing, in particular loss of firmness, loss of elasticity, reduction in thickness of the skin, and the appearance of wrinkles and/or fine lines.

The invention also relates to a cosmetic method for care and/or make-up of keratin materials, in particular the skin and/or the lips, comprising the topical application on said keratin materials, of a cosmetic composition according to the invention.

The invention also relates to an extract of Dendrobium fimbriatum, characterised in that it is obtained by an extraction method comprising at least the steps of:
a) grinding the plant material;
b) at least one extraction step followed by a filtration step;
c) concentrating the extract obtained at the end of step b);
d) adding butylene glycol, pentylene glycol, potassium sorbate/sodium benzoate, glycerol or propanediol, preferably propanediol;
e) filtering, and
f) obtaining an extract of Dendrobium fimbriatum.

The invention also relates to an extract of Dendrobium fimbriatum, characterised in that it comprises at least one metabolite chosen from betaine and cis and trans melilotoside.

The invention also relates to a method for obtaining an extract of Dendrobium fimbriatum comprising at least the steps of:
a) grinding the plant material;
b) at least one extraction step followed by a filtration step;
c) concentrating the extract obtained at the end of step b);
d) adding butylene glycol, pentylene glycol, potassium sorbate/sodium benzoate, glycerol or propanediol, preferably propanediol;
e) filtering, and
f) obtaining an extract of Dendrobium fimbriatum.

### DETAILED DESCRIPTION OF THE INVENTION

No-Rinse Cosmetic Composition Comprising an Extract of Dendrobium fimbriatum

In its first aspect, the invention relates to a no-rinse cosmetic composition for topical application to keratin materials, in particular on the skin and/or the lips and in particular the skin of the face and/or of the neck, comprising an extract of Dendrobium fimbriatum. “Dendrobium fimbriatum” in the context of the invention means a plant from the orchid family (Orchidaceae) and originating from South-East Asia It is also known under the names Callista fimbriata, Dendrobium normale, Dendrobium fimbriatum var. oculatum, Dendrobium paxtonii, Callista oculata. In the rest of the description, reference will be made to extract of Dendrobium fimbriatum or of Dendrobium fimbriatum var. oculatum. According to the present invention, the extract of Dendrobium fimbriatum is used, in particular, in no-rinse cosmetic compositions as cosmetic active ingredient and in particular as “anti-ageing” cosmetic active ingredient.

The extract of Dendrobium fimbriatum according to the invention is a plant extract. The term “plant extract” means, in general, an isolated substance obtained by extraction from a plant raw material, and which does not pre-exist in nature as such.

The term “plant material” or “plant raw material” means all or part of the plant Dendrobium fimbriatum from which the extract of Dendrobium fimbriatum according to the invention is prepared.

The extract of Dendrobium fimbriatum according to the invention can, in particular, be prepared from a plant material chosen from the roots, stems, flowers, petals and the mixtures thereof or from the entire plant. The extract of Dendrobium fimbriatum according to the invention is preferably prepared from the entire plant.

Prior to the extraction step itself, the plant material can be dried, lyophilised and/or ground. Alternatively, the plant material can be used wet then ground. According to a particular embodiment, the plant material is ground before extraction, for example by means of a mortar, a mixer, a traditional mill, or any other method conventionally used in the field and known to a person skilled in the art.

According to the invention, the extract of Dendrobium fimbriatum is a polar extract, advantageously obtained by means of a cosmetically-acceptable polar solvent. The term “cosmetically-acceptable” means compatible with the keratin materials, in particular the skin and/or the lips.

The preferred polar solvents are those consisting of a compound comprising at least one O—H polar covalent bond. A particularly preferred polar solvent is chosen as a solvent or mixture of solvents from water, C1-C4 alcohols, such as ethanol, glycols, such as ethylene glycol, glycerol, butylene glycol and propylene glycol, pentylene glycol, potassium sorbate/sodium benzoate, propanediol and the mixtures thereof.

According to a preferred embodiment, the extract of Dendrobium fimbriatum is a hydroalcoholic extract, obtained by extraction of the plant material in a mixture of water and alcohol, the alcohol representing 1% to 99% v/v (volume/volume) of the water/alcohol mixture. By way of non-limiting examples, the plant material can be extracted using a mixture comprising 10% v/v water and 90% v/v alcohol; 20% v/v water and 80% v/v alcohol; 30% v/v water and 70% v/v alcohol; 40% v/v water and 60% v/v alcohol; 50% v/v water and 50% v/v alcohol; 60% v/v water and 40% v/v alcohol; 70% v/v water and 30% v/v alcohol; 80% v/v water and 20% v/v alcohol; 90% v/v water and 10% v/v alcohol. According to a preferred embodiment the plant material is extracted using a mixture comprising 50% v/v water and 50% v/v alcohol.

The extract of Dendrobium fimbriatum is preferably obtained by extracting the plant material in a water and ethanol mixture, in particular a mixture comprising between 45% and 55% v/v water and between 45 and 55% v/v ethanol, preferably 50% v/v water and 55% v/v ethanol.

The extract of Dendrobium fimbriatum according to the invention can be prepared by various extraction methods known to a person skilled in the art, implementing steps of grinding the plant material, dispersing the ground material in a polar solvent, separating the soluble and insoluble phases by filtration, concentrating and optionally re-dissolving. According to the invention, the extraction can be carried out hot by reflux or else by maceration at ambient temperature. The extraction can also be carried out using well-known physical chemistry techniques such as, in particular, ultrasound, microwaves, extrusion, pulsed electric field, sub-critical water and/or cryo-extraction.

The extraction of the plant material is preferably carried out hot by reflux. The term “hot by reflux” means that the extraction temperature is at least 60° C., preferably at least 65° C., more preferably at least 70° C., preferably at least 75° C., more preferably at least 80° C. In the case of an extraction by reflux, the duration of the extraction can range, in particular, from 15 to 180 minutes, preferably 30 to 120 minutes, preferably 45 to 90 minutes, 50 to 70 minutes, being for example 50, 55, 60, 65 or 70 minutes.

The extraction method advantageously comprises a filtration step for separating the liquid phase from the plant material. A person skilled in the art will choose suitable filtration sieves, in particular with filtration diameters of 0.1 μm to 0.5 μm, in particular 0.2 to 0.3 μm.

The cycle of extraction then filtration can be repeated several times in order to deplete the plant material of substances having an affinity for the extraction solvent. In particular, the extraction/filtration cycle can be repeated at least twice and preferably at least three times, or even four or more times, such as five times for example.

At the end of each extraction/filtration cycle, the solvent is collected and replaced by as yet unused solvent, with a view to repeating the extraction step. The successive repetition of the extraction steps advantageously enables extraction of a maximum of compounds of interest from the plant material to be extracted, in particular by avoiding the solvent becoming saturated with molecules of interest.

At the end of the successive extraction steps, the various collected solvents are then combined.

Once the plant material is depleted of substances having an affinity for the extraction solvent, the latter can be eliminated. Alternatively, it can be kept for other uses.

The extract can advantageously be concentrated by removing a portion of the extraction solvent. It is also possible to obtain an aqueous concentrate devoid of a significant quantity of organic solvents, or even to obtain a dry residue by eliminating all the extraction solvent. Alternatively, the product from the extraction step can be lyophilised or atomised in order to be in the form of a powder.

The solvents collected at the end of each extraction/filtration cycle are preferably combined, the extract is vacuum concentrated and then the ethanol is at least partially evaporated. Finally, the concentrated extract is then diluted in propanediol then filtered before packaging. A person skilled in the art will choose suitable filtration sieves, in particular with filtration diameters of 0.1 μm to 0.5 μm, in particular 0.2 μm.

The term “packaging” means packing the obtained extract under conditions enabling its preservation, storage and handling.

Thus, the present invention also relates to an extract of Dendrobium fimbriatum characterised in that it is obtained by an extraction method comprising at least the steps of:
a) grinding the plant material;
b) at least one extraction step followed by a filtration step;
c) concentrating the extract obtained at the end of step b);
d) adding butylene glycol, pentylene glycol, potassium sorbate/sodium benzoate, glycerol or propanediol, preferably propanediol;
e) filtering, and
f) obtaining an extract of Dendrobium fimbriatum.

According to a particular embodiment of the invention, the extract of Dendrobium fimbriatum is obtained according to an extraction method comprising at least the following steps:
a) grinding the plant material, preferably the entire plant, dry;
b) extraction by reflux using a hydroalcoholic solvent;
c) filtering to recover the grounds and the extract;
d) optionally, 1 to 4 repetitions of steps b) and c) starting with the grounds collected at the end of the preceding extraction/filtration step and recovery of a new extract at the end of each extraction/filtration step;
e) combining the extract collected at the end of each extraction/filtration step;
f) evaporating a portion of the extraction solvent and vacuum concentration of the extract;
g) adding butylene glycol, pentylene glycol, potassium sorbate/sodium benzoate, glycerol or propanediol, preferably propanediol;
h) filtering, and
i) packaging the extract of Dendrobium fimbriatum.

Preferably a mixture of water and ethanol is used as hydroalcoholic solvent, in particular a mixture comprising between 45% and 55% v/v water and between 45 and 55% v/v ethanol, preferably 50% v/v water and 50% v/v ethanol.

The extraction of the plant material is preferably carried out hot by reflux, in particular at a temperature of at least 60° C., preferably at least 65° C., more preferably at least 70° C., preferably at least 75° C., more preferably at least 80° C.

The duration of the extraction for each extraction/filtration cycle is preferably between 15 and 180 minutes, preferably between 30 and 120 minutes, preferably between 45 and 90 minutes, between 50 and 70 minutes, being for example 50, 55, 60, 65 or 70 minutes.

The filtration steps preferably use filtration sieves with filtration diameters of 0.1 μm to 0.5 μm, in particular 0.2 to 0.3 μm.

According to a particular and preferred embodiment of the invention, the extract of Dendrobium fimbriatum is obtained according to an extraction method comprising at least the following steps:
a) grinding of the dry vegetation, preferably the entire plant;
b) extraction by reflux with 50% ethanol at 80° C. for 1 hour;
c) filtering to recover the grounds 1 and the extract X;
d) extracting the grounds 1 by reflux with 50% ethanol at 80° C. for 1 hour;
e) filtering to recover the grounds 2 and the extract Y;
f) extraction of the grounds by reflux with 50% ethanol at 80° C. for 1 hour;
g) filtering to recover the grounds 3 and the extract Z;
h) combining the extracts X, Y and Z;
i) evaporating the ethanol and vacuum concentrating the extract;
j) adding propanediol;
k) filtering, and
l) packaging the extract of Dendrobium fimbriatum.

The extract of Dendrobium fimbriatum attained according to the extraction method of the invention has the INCI name: WATER (and) PROPANEDIOL (and) DENDROBIUM FIMBRIATUM OCULATUM EXTRACT.

The Dendrobium fimbriatum extract according to the invention, in the form of a solution, preferably comprises approximately 1% dry matter, approximately 35% propanediol and approximately 64% water. The percentages given are percentages by weight relative to the total weight of the extract. The term “approximately” means that the proportions measured can vary by +/−10%, preferably +/−5% and more preferably +/−2%.

The Dendrobium fimbriatum extract according to the invention, in particular obtained according to the method described above, preferably comprises a total mineral content ranging from 300 to 1000 ppm (in particular phosphorus, magnesium, calcium, sodium and potassium), measured by method EN ISO 17294-2—ICP-MS.

The extract of Dendrobium fimbriatum according to the invention preferably comprises sugars with a content ranging from 0.1% to 0.5%, measured by the method using anthrone (Loewus, 1952).

The extract of Dendrobium fimbriatum preferably comprises polyphenols with a content ranging from 0.1% to 0.5%, measured by the Folin Ciocalteu method (Ainsworth, 2007). According to a preferred embodiment, the extract of Dendrobium fimbriatum obtained by the method described above, is characterised in that it comprises at least one metabolite chosen from betaine and cis and trans melilotoside.

The invention therefore likewise relates to an extract of Dendrobium fimbriatum, characterised in that it comprises at least one metabolite chosen from betaine and cis and trans melilotoside.

According to a preferred embodiment, the extract of Dendrobium fimbriatum used in the cosmetic compositions of the invention is obtained according to an extraction method as described above.

The extract of Dendrobium fimbriatum used in the cosmetic compositions of the invention is preferably obtained according to an extraction method comprising at least the following steps:
a) grinding of the dry vegetation, preferably the entire plant;
b) extraction by reflux with 50% ethanol at 80° C. for 1 hour;
c) filtering to recover the grounds 1 and the extract X;
d) extracting the grounds 1 by reflux with 50% ethanol at 80° C. for 1 hour;
e) filtering to recover the grounds 2 and the extract Y;
f) extraction of the grounds by reflux with 50% ethanol at 80° C. for 1 hour;
g) filtering to recover the grounds 3 and the extract Z;
h) combining the extracts X, Y and Z;
i) evaporating the ethanol and vacuum concentrating the extract;
j) adding butylene glycol, pentylene glycol, potassium sorbate/sodium benzoate, glycerol or propanediol, preferably propanediol;
k) filtering, and
l) packaging the extract of Dendrobium fimbriatum.

According to another embodiment, the extract of Dendrobium fimbriatum used in the cosmetic compositions of the invention is characterised in that it comprises at least one metabolite chosen from betaine and cis and trans melilotoside, in particular betaine.

The invention likewise relates to a method for obtaining an extract of Dendrobium fimbriatum comprising at least the steps of:
a) grinding the plant material;
b) at least one extraction step followed by a filtration step;
c) concentrating the extract obtained at the end of step b);
d) adding butylene glycol, pentylene glycol, potassium sorbate/sodium benzoate, glycerol or propanediol, preferably propanediol;
e) filtering, and
f) obtaining an extract of Dendrobium fimbriatum.

According to a particular embodiment of the invention, the method for obtaining an extract of Dendrobium fimbriatum comprises at least the following steps:
a) grinding the dry plant material, preferably the entire plant;
b) extraction by reflux using a hydroalcoholic solvent;
c) filtering to recover the grounds and the extract;
d) optionally, 1 to 4 repetitions of steps b) and c) starting with the grounds collected at the end of the preceding extraction/filtration step and recovery of a new extract at the end of each extraction/filtration step;
e) combining the extract collected at the end of each extraction/filtration step;
f) evaporating the extraction solvent and vacuum concentration of the extract;
g) adding butylene glycol, pentylene glycol, potassium sorbate/sodium benzoate, glycerol or propanediol, preferably propanediol;
h) filtering, and
i) packaging.

Preferably a mixture of water and ethanol is used as hydroalcoholic solvent, in particular a mixture comprising between 45% and 55% v/v water and between 45 and 55% v/v ethanol, preferably 50% v/v water and 55% v/v ethanol.

The extraction of the plant material is preferably carried out hot by reflux, in particular at a temperature of at least 60° C., preferably at least 65° C., more preferably at least 70° C., preferably at least 75° C., more preferably at least 80° C.

The duration of the extraction for each extraction/filtration cycle is preferably between 30 and 120 minutes, preferably between 45 and 90 minutes, between 50 and 70 minutes, being for example 50, 55, 60, 65 or 70 minutes.

The filtration steps preferably use filtration sieves with filtration diameters of 0.1 μm to 0.5 μm, in particular 0.2 μm.

According to a particular and preferred embodiment of the invention, the extract of Dendrobium fimbriatum is obtained according to an extraction method comprising at least the following steps:
a) grinding of the dry vegetation, preferably the entire plant;
b) extraction by reflux with 50% ethanol at 80° C. for 1 hour;
c) filtering to recover the grounds 1 and the extract X;
d) extracting the grounds 1 by reflux with 50% ethanol at 80° C. for 1 hour;
e) filtering to recover the grounds 2 and the extract Y;
f) extraction of the grounds by reflux with 50% ethanol at 80° C. for 1 hour;
g) filtering to recover the grounds 3 and the extract Z;
h) combining the extracts X, Y and Z;
i) evaporating the ethanol and vacuum concentrating the extract;
j) adding butylene glycol, pentylene glycol, potassium sorbate/sodium benzoate, glycerol or propanediol, preferably propanediol;
k) filtering, and
l) packaging the extract of Dendrobium fimbriatum.

Dosage

According to the invention, the extract of Dendrobium fimbriatum is used in cosmetic compositions in a quantity that is effective for obtaining the desired effect. In a particular embodiment, the extract of Dendrobium fimbriatum is present in said cosmetic composition with a content ranging from 0.01% to 10%, preferably ranging from 0.1% to 10%, in particular from 0.2% to 5%, preferably from 0.3% to 3% by weight of raw material relative to the total weight of the composition, corresponding to a content ranging from 0.0001% to 0.1%, preferably 0.002% to 0.05%, more preferably 0.003% to 0.03% by weight of dry matter relative to the total weight of the composition.

In a particular and preferred embodiment, the Dendrobium fimbriatum extract used in the cosmetic compositions of the invention is the product marketed under the name FINGE GOLD ORCHID.

In a preferred embodiment, said cosmetic composition comprising the Dendrobium fimbriatum extract is a composition for care and/or make-up of keratin materials, in particular of the skin and/or lips and in particular of the skin of the face and/or of the neck. According to certain embodiments, in addition to the extract of Dendrobium fimbriatum, the cosmetic composition of the invention can also comprise at least one other plant extract, for example an extract of an orchid of the species Gastrodia elata, and preferably the extract with INCI name: GASTRODIA ELATA ROOT EXTRACT. The Gastrodia elata extract is present in the compositions of the invention in an effective quantity for obtaining the desired effect, in particular for preventing and/or reducing the signs of skin ageing, in particular loss of firmness, loss of elasticity, reduction in thickness of the skin, and the appearance of wrinkles and/or fine lines. In particular, said Gastrodia elata extract is present with a content ranging from 0.01% to 10%, in particular 0.02% to 5%, preferably 0.05% to 4% by weight of raw material (weight of product) relative to the total weight of the composition, corresponding to a content ranging from 0.0001% to 0.1%, preferably 0.0002% to 0.05%, more preferably 0.0005% to 0.04% by weight of dry matter relative to the total weight of the composition.

According to certain embodiments, the cosmetic composition of the invention comprises at least the extract of Dendrobium fimbriatum according to the invention and in addition one or more cosmetically acceptable excipients from those known to a person skilled in the art, with a view to obtaining a composition for topical application, for example in the form of a milk, cream, pomade, water-in-oil or oil-in-water emulsion, salve, stick, gel, lotion, serum, or else powder.

According to the nature of the composition, one or more cosmetically acceptable excipients will be chosen from polymers, surfactants, rheological agents, perfumes, electrolytes, pH modifiers, antioxidants, preservatives, dyes, nacres, pigments and the mixtures thereof.

The cosmetic composition of the invention may be in any dosage form suitable for topical application on keratin materials, in particular the skin and/or lips and, in particular, the skin of the face and/or neck, further comprising the extract of Dendrobium fimbriatum according to the invention, and at least one cosmetic adjuvant chosen from the group consisting of antioxidants, emollients, moisturisers, anti-ageing agents, perfumes and the mixtures thereof

Cosmetic Uses and Methods

The cosmetic methods and cosmetic uses described below are intended for an application on keratin materials, in particular healthy keratin materials, preferably healthy skin and/or lips and, in particular, healthy lips (“healthy” subjects).

The term “healthy keratin materials” according to the invention means keratin materials not exhibiting diseases or disorders which would be part of a pathological condition (“unhealthy” subjects, affected by a pathology). In the rest of the description, healthy skin and/or lips or skin and/or lips will be referred to interchangeably.

As illustrated in the examples below, the extract of Dendrobium fimbriatum according to the invention is capable of significantly increasing the synthesis of several proteins of the extracellular matrix, in particular elastin, fibrillin 1, collagen 1 and nidogen. The extract of

Dendrobium fimbriatum according to the invention therefore has a cosmetic interest, in particular as a cosmetic “anti-ageing” active ingredient.

The term cosmetic “anti-ageing” active ingredient means that the Dendrobium fimbriatum extract according to the invention is effective for preventing and/or reducing the signs of skin ageing, in particular loss of firmness, loss of elasticity, reduction in thickness of the skin, and appearance of wrinkles and/or fine lines, increasing cellular longevity.

Hence, the invention also relates to a non-therapeutic cosmetic use of an extract of Dendrobium fimbriatum as described above, as an agent for preventing and/or reducing the signs of skin ageing, in particular loss of firmness, loss of elasticity, reduction in thickness of the skin, and the appearance of wrinkles and/or fine lines.

According to a particular and preferred embodiment, the invention relates to the use of an extract of Dendrobium fimbriatum, characterised in that it is marketed under the name FINGE GOLD ORCHID.

According to a preferred embodiment of the invention, the invention relates to the use of an extract of Dendrobium fimbriatum, characterised in that it is obtained by an extraction method comprising at least the steps of:
a) grinding the plant material;
b) at least one extraction step followed by a filtration step;
c) concentrating the extract obtained at the end of step b);
d) adding propanediol;
e) filtering, and
f) obtaining an extract of Dendrobium fimbriatum.

According to a preferred embodiment, the invention relates to the use of an extract of Dendrobium fimbriatum, characterised in that it comprises at least one metabolite chosen from betaine and cis and trans melilotoside, in particular betaine.

The present invention likewise relates to a cosmetic method for care and/or make-up of keratin materials, in particular the skin and/or lips, comprising the topical application on said keratin materials, of a cosmetic composition as described above.

According to another particular embodiment, the cosmetic composition used in the cosmetic uses or methods of the invention further comprises an extract of orchid of the species Gastrodia elata, and preferably the extract with INCI name: GASTRODIA ELATA ROOT EXTRACT.

According to the present invention, the cosmetic method is intended to prevent and/or reduce the signs of skin ageing, in particular loss of firmness, loss of elasticity, reduction in thickness of the skin, and the appearance of wrinkles and/or fine lines.

The cosmetic composition is preferably applied on skin that is aged or exhibiting signs of ageing.

The invention will now be illustrated in the following non-limiting examples. The percentages (%) are expressed by weight of raw material (RM) relative to the total weight of the composition, unless otherwise indicated.

### EXAMPLES

### Example 1: Preparing an Extract of Dendrobium fimbriatum

Step 1: grinding the entire dry Dendrobium fimbriatum plant;
Step 2: extraction by reflux with 50% ethanol at 80° C., for 1 hour;
Step 3: filtration, enabling recovery of the grounds 1 and the extract X;
Step 4: extraction of the grounds 1 by reflux with 50% ethanol at 80° C., for 1 hour;
Step 5: filtration enabling recovery of the grounds 2 and the extract Y;
Step 6: extraction of the grounds 2 by reflux with 50% ethanol at 80° C., for 1 hour;
Step 7: filtration, enabling recovery of the grounds 3 and the extract Z;
Step 8: combination of extracts X, Y and Z;
Step 9: evaporation of the ethanol and vacuum concentration of the extract;
Step 10: addition of propanediol;
Step 11: filtration, and
Step 12: packaging of the extract of Dendrobium fimbriatum.

The extract thus obtained has the INCI name: WATER/AQUA, PROPANEDIOL DENDROBIUM FIMBRIATUM OCULATUM EXTRACT. Its dry matter content is 1%, it comprises 35% and 64% water.

### Example 2: Phytochemical Characterisation of the Dendrobium fimbriatum Extract Prepared According to Example 1

In this study, the Applicant has studied the phytochemical profile of a Dendrobium fimbriatum extract obtained by the method of example 1.

2.1. Fractionation of the Extracts by Centrifugal Partition Chromatography (CPC)

Two CPC experiments were performed successively.

2.1.1. CPC 1

The dry extract, dissolved beforehand in a stationary phase/mobile phase mixture, was injected in CPC into a previously equilibrated column (FCPE300® (Rousselet Robatel Kromaton)). The flow rate was 20 mL/min and rotational speed of the column was 1200 rpm. The three-phase solvent system corresponds to n-heptane/methyl-ter-butyl-ether/acetonitrile/water (1/1/1/1, v/v) and the separation is carried out in ascendant mode. Fractions of 20 ml were collected throughout the experiment (elution and extrusion) and combined according to their thin-layer chromatography profiles. Four fractions were thus obtained.

All the compounds retained inside the column were recovered by extrusion and vacuum dried in order to obtain the CPC 2 injection.

2.1.2. CPC2

The dry extract, dissolved beforehand in a stationary phase/mobile phase mixture, was injected in CPC into a previously equilibrated column (FCPE300® (Rousselet Robatel Kromaton)). The flow rate was 20 mL/min and rotational speed of the column was 1200 rpm. The three-phase solvent system corresponds to n-heptane/methyl-ter-butyl-ether/acetonitrile/water (1/1/1/1, v/v) and the separation is carried out in ascendant mode. Fractions of 20 ml were collected throughout the experiment (elution and extrusion) and combined according to their thin-layer chromatography profiles. Nine fractions were thus obtained.

2.2. NMR Analyses and Identification of the Main Metabolites

An aliquot of each fraction, F1 to F13, was dissolved in DMSO-d6 and analysed by 13C NMR at 298 K on a Bruker Avance AVIII-600 spectrometer (Karlsruhe, German) equipped with a cryoprobe. After processing the spectra, the absolute intensities of all the 13C NMR signals were collected automatically and grouped in the spectra of the series of fractions, using a computer script developed by Natexplore. The resulting table was subjected to a hierarchical group analysis (PermutMatrix, 1.9.3, LIRMM, Montpellier, France). The resulting 13C NMR chemical shift clusters were viewed in the form of dendrograms on a two-dimensional map. In order to identify metabolites, each 13C NMR chemical shift cluster obtained by HCA was manually submitted to the structure search engine of the ACD/NMR Workbook Suite 2012 (ACD/Labs, Ontario, Canada) database management software comprising the structures and predicted chemical shifts for low molecular weight natural products. 2D NMR experiments (HSQC, HMBC and COSY) were carried out on fractions containing supposedly identified compounds in order to confirm the molecular structures proposed by the database at the end of the dereplication process.

The main metabolites identified by NMR in each fraction are presented in table 1.

TABLE 1
Mass and overall composition of the fractions of the CPC.

| CPC | Mass | % crude | |
|---|---|---|---|
| fraction | (mg) | extract | Composition |
| Eluate 1 | 26 | 1.3% | Mixture of aliphatic acids |
| Eluate 2 | 21 | 1.0% | (poly-unsaturated and saturated fatty acids or |
| Eluate 3 | 14 | 0.7% | alcohols) |
| Eluate 4 | 16 | 0.8% | |
| Eluate 5 | 112 | 5.6% | Melilotic acid (Med); Coumarin (Min−−); ortho- |
| | | | coumaric acid (Min); Dendrophenol (Min) |
| Eluate 6 | 135 | 6.7% | Melilotic acid (Maj); Coumarin (Min); ortho-coumaric |
| | | | acid (Min); Dendrophenol (Min) |
| Eluate 7 | 51 | 2.5% | Melilotic acid (Med); Coumarin (Min−−); ortho- |
| | | | coumaric acid (Min); Dendrophenol (Min); |
| | | | Syringaldehyde (Min); vanillic acid (Min); p- |
| | | | hydroxybenzoic acid (Min) |
| Eluate 8 | 59 | 2.9% | 3-Hydroxy-3-(2-hydroxyphenyl)-propanoic acid (Maj) |
| Eluate 9 | 28 | 1.4% | Levulinic acid (Maj); suberic acid (Med); 2,4- |
| | | | dimethyl-5-oxotetrahydrofuran-3-carboxylic acid |
| | | | (Min); 1,3,4-pentanetricarboxylic acid (Min) |
| Eluate 10 | 51 | 2.5% | Dihydromelilotoside (Min−) |
| Eluate 11 | 142 | 7.1% | Dihydromelilotoside (Maj); trans-melilotoside (Min); |
| | | | cis-melilotoside (Min) |
| Eluate 12 | 1321 | 65.7% | Hexitol (Maj); Glycerol (Maj); Choline (Min); Betaine |
| | | | (Min) |
| Eluate 13 | 35 | 1.7% | Dihydromelilotoside (Min−); trans-melilotoside (Min); |
| | | | cis-melilotoside (Min); Adenosine (Min); Uridine |
| | | | (Min) |

Maj = major;
Med = medium;
Min = minor

2.3. Conclusion

The crude extract was fractionated by the CPC technique into a total of 13 fractions during two successive experiments. The least polar fractions F1-F4 (representing approximately 3.8% of the mass of the crude extract) were composed of long chain alkyls (fatty acids or alcohols). The moderately polar fractions F5-F10 (representing approximately 21.6% of the mass of the crude extract) contained a diversity of polyphenols (melilotic acid, dendrophenol, dihydromelilotoside), as well as coumarin, syringaldehyde and several phenolic acids such as ortho-coumaric acid, vanillic acid, p-hydroxybenzoic acid and 3-hydroxy-3-(2-hydroxyphenyl)-propanoic acid. Other non-phenolic acids were also identified in these fractions (levulinic acid, suberic acid, 2,4-dimethyl-5-oxotetrahydrofurane-3-carboxylic acid and 1,3,4-pentanetricarboxylic acid). The most polar fractions F11-F13 recovered during the extrusion step (representing approximately 74.5% of the mass of the crude extract), were mainly composed of simple sugars, as well as dihydromelotoside, trans-melilotoside, cis-melilotoside, glycerol, choline, betaine, uridine and adenosine.

The extract thus obtained and described was tested for its biological effects in the following examples.

### Example 3: Effect of Dendrobium fimbriatum on the Expression of Dermal Extracellular Matrix Markers in Normal Human Fibroblasts

In this study, the Applicant wished to evaluate the effect of the extract of the orchid Dendrobium fimbriatum prepared according to example 1, on the expression of various proteins involved in the synthesis of the extracellular matrix (ECM) in normal human fibroblasts in culture (NHF).

3.1. Culture and Treatment of Normal Human Fibroblasts

The Normal Human Fibroblasts (NHF) came from an abdominoplasty of a 37-year-old female donor. The cells were seeded in DMEM 1 g/L glucose medium supplemented with 10% foetal calf serum at passage 5 (P5), with a seeding density of 15,000 cells per well, in 96-well plates.

The dry extract of Dendrobium fimbriatum was used at a concentration of 50 μg/ml in ethanol.

At confluence (D1), the NHF were treated with the extract described above in DMEM 1 g/L glucose medium+10% FCS (foetal calf serum). The treatment was carried out in n=4 for each condition, for 5 days with re-treatment of the monolayer at D3. For the control (untreated) conditions, the medium was replaced by fresh culture medium.

3.2. Immunomarking

3.2.1. Marking Protocol

At the end of the treatments, the cells were rinsed three times with PBS (PBS Tablets, Invitrogen GIBCO), then fixed with formalin (Formalin Solution 10% Neutral Buffered, Sigma) for 10 minutes.

After rinsing 3 times with PBS, the cell membranes were permeabilised or not (depending on the marker studied) with a solution of 0.1% PBS Triton (Triton X-100, Sigma). They were then rinsed again 3 times with PBS.

The cells were covered with a 1% PBS/BSA solution (Albumin, from bovine serum, Sigma), for 30 minutes at ambient temperature.

The 1% PBS/BSA solution was then replaced by a primary antibody solution corresponding to each protein marked (see table 1) diluted in the 1% PBS/BSA. The plates were then incubated overnight at 4° C.

The cells were then rinsed three times with PBS and covered with a secondary antibody solution according to the primary antibody to be targeted (see table 1) and DAPI at 1/1000th (4′,6′-diamidino-2-phenylindole, dihydrochloride, Invitrogen Molecular Probes) diluted in the 1% PBS/BSA. The plates were kept for one hour in the dark at ambient temperature.

The secondary antibody solution was then sucked up and the cells rinsed twice with PBS and once with distilled water. 1 mL of PBS was deposited in each well. The plates were kept at 4° C. and in the dark, until the images were acquired.

TABLE 2
List of primary antibodies and conditions of use

| Name of | | | Permeabil- | | |
|---|---|---|---|---|---|
| target | Supplier | Reference | isation | Dilution | Species |
| Elastin | Abcam | Ab 23747 | − | 1/200 | Rb |
| Fibrillin 1 | Thermo | MA12770 | − | 1/200 | Ms |
| Collagen 1 | Novotec | 20111 | − | 1/200 | Rb |
| Nidogen 1 | R&D system | AF2570 | + | 1/200 | Goat |

3.2.2. Image Acquisition by HCS

The plates were scanned with the ArrayScan XTi from Thermo Cellomics.

Acquisition conditions:
Detection: DAPI: filter XF53_386_23
Alexa Fluor 568: filter XF53_572_15
   Resolution: 1104×1104
   Objective: 10× dry
   Number of images: 25 per well

3.2.3. Image Analysis

The images with then analysed using the analysis software of the Array Scan system by the Spot detector bio-application.

This program (pipeline) can detect the red marking of the targeted protein, corresponding to its expression. The surface of the measurement zone is defined as being the entire surface of the image.

The number of cells was determined by counting the nuclei through detection of the blue marking of DAPI.

According to the markers and their cellular expression, the results can be expressed by:
Intensity of the marking detected (Threshold)/No. of cells: this method was used to quantify the structures in the form of fibres, for example.
Intensity of the marking in the total field/No. of cells: this method provided a general quantification of the synthesis of the studied marker without taking account of the structure.

3.3. Statistical Analyses

The statistical analysis of results consists in calculating the average, the standard deviation and the confidence interval (α=0.05).

The significance of the differences in these ratios observed between the control and the treated samples was measured by the Student test. The Student test is said to be significant if its value is less than 0.05.

3.4. Results

TABLE 3

| | D. fimbriatum 50 μg/mL |
|---|---|
| Elastin | +96% |
| Fibrillin 1 | +35% |
| Collagen 1 | +41% |
| Nidogen | +52% |

The results show that the treatment of NHF by the extract of Dendrobium fimbriatum according to the invention has induced a very strong stimulation on the expression of elastin (+96%). It also induced a strong stimulation on the expression of other ECM markers such as fibrillin 1, collagen 1 and nidogen (+35% to +52%).

Taken altogether, these results suggest that the extract of Dendrobium fimbriatum according to the invention is capable of significantly increasing the synthesis of several components of the extracellular matrix of the skin environment. This extract therefore has a cosmetic interest for use in connection with the physiological functions of the skin, in particular preventing and/or preventing loss of firmness, loss of elasticity of the skin, reduction in thickness of the skin, and/or the appearance of wrinkles and/or fine lines.

### Example 4: Effect of Dendrobium fimbriatum or Gastrodia elata Extracts on Longevity of NHF

In this study, the Applicant wished to evaluate the effect of the extract of orchid Dendrobium fimbriatum prepared according to example 1, on the longevity of normal human fibroblasts.

4.1 Study Protocol

4.1.1. Model of Senescent Fibroblasts Studied by Flow Cytometry

The fibroblasts coming from the dermis of a donor (58-year-old female donor) were cultured and aged by a series of passages which gave them an aged phenotype (model of senescent fibroblasts).

Certain morphological markers of senescence were measured by flow cytometry. This technique advantageously makes it possible to study, at very high flow rate and in a quantitative manner, the optical characteristics (fluorescence, diffusion of light linked to cellular morphology, etc.) of a large number of cells (several tens of thousands). It is therefore possible to derive statistically significant data.

The cells to be studied were placed in suspension in a medium. In the cytometer, the cells are sent one by one into an analysis chamber where they are subjected to a plurality of laser beams which can measure or evaluate cellular parameters.

The diffracted light measured opposite the laser beam is used to evaluate the size of the cells (forward scatter parameter, FSC). The diffracted light measured on the side (Side scatter parameter, SSC) gives a measure of the granularity of the cell. This granularity corresponds to the complexity of the cell, for example the density of organelles, protein aggregates, and internal or surface irregularities. With senescence, the size and granularity of the cells increase, in particular following oxidative damage on the organelles, the accumulation of lipofuscin, or even damage to the macromolecules.

4.1.2. Culture and Treatment of Normal Human Fibroblasts

The effect of the Dendrobium fimbriatum extract according to the invention was evaluated comparatively with that of an extract of orchid root of the species Gastrodia elata.

Dendrobium fimbriatum Extract:
Parent solution: 1% (10 mg/mL of dry extract) in a (50:50) EtOH/H2O mixture
Final solution: 0.0006%, i.e. 6 μg/mL (diluted in the culture medium at 1/1666.6)

Gastrodia elata Extract:
Parent solution: 100% BG (butylene glycol)/H2O (50:50) containing 1% root extract
Final solution: 0.06% (diluted in the culture medium at 1/1666.7)

Treatment Conditions:
Not treated: 10% (FCS)
Ctl Exp BG/H₂O (50:50) 1/400
Gastrodia elata 0.06% (corresponding to 6 μg/mL of dry extract)
Dendrobium fimbriatum 6 μg/mL of dry extract

The senescent fibroblasts obtained in paragraph 4.1.1. were seeded in the DMEM 1 g/L glucose medium supplemented by 10% FCS and treated under the conditions described above at D1, D4, D6, D8, D11 and D13.

At D14 the cells were washed and recovered by treatment with Accutase® then the morphological senescence markers were measured by flow cytometry.

4.2. Results

For each of the parameters, FSC and SCC, 4 conditions were compared, 14 days after placing the cells in contact with the ingredients:
(1): untreated cells at an early passage (Passage 7)
(2): untreated cells are delayed passage (Passage 28)=aged cells
(3): cells at a late passage (P28) treated with Gastrodia elata (eq. 6 μg/mL dry extract)
(4): cells at a late passage (P28) treated with Dendrobium fimbriatum (6 μg/mL dry extract)

4.2.1. Measurement of FSC

TABLE 4

| Condi- | Pas- | Ingre- | | | |
|---|---|---|---|---|---|
| tion | sages | dient | FSC | Comparison | p |
| (1) | 7 | NT | 337056 +/− 9714 | — | |
| (2) | 28 | NT | 423576 +/− 10297 | +27.5% vs. (1) | <0.001 |
| (3) | 28 | GE | 355221 +/− 3842 | −16.2% vs. (2) | <0.001 |
| (4) | 28 | DF | 361729 +/− 3887 | −14.6% vs. (2) | =0.001 |

The size of the cells is significantly higher in the aged fibroblasts. The treatment with Dendrobium fimbriatum reduces this morphological sign of cellular senescence.

4.2.2. Measurement of SSC

TABLE 5

| Condi- | Pas- | Ingre- | | | |
|---|---|---|---|---|---|
| tion | sages | dient | SSC | Comparison | p |
| (1) | 7 | NT | 557682 +/− 13918 | — | |
| (2) | 28 | NT | 704152 +/− 31509 | +26.3% vs. (1) | =0.002 |
| (3) | 28 | GE | 684407 +/− 8569 | −2.8% vs. (2) | ns |
| (4) | 28 | DF | 651868 +/− 10784 | −7.3% vs. (2) | =0.053 |

The granularity of the cells is significantly higher in the aged fibroblasts. The treatment with Dendrobium fimbriatum reduces this morphological sign of cellular senescence, whereas the Gastrodia elata extract shows no effect.

The results therefore suggest that the treatment with Dendrobium fimbriatum extract according to the invention had a benefit for combating senescence of the fibroblasts. This effect is notably better than for the extract of Gastrodia elata.

Finally, this extract can be used as an anti-ageing active ingredient.

### Example 5: Cosmetic Formulations

5.1. Composition in the Form of a Cream

TABLE 6

| Ingredients | % by weight |
|---|---|
| AQUA (WATER) | QSP100 |
| CAPRYLIC/CAPRIC TRIGLYCERIDE | 20.000 |
| GLYCERINE | 5.000 |
| PENTYLENE GLYCOL | 3.000 |
| GLYCERYL STEARATE | 1.500 |
| ESTERS of JOJOBA | 1.000 |
| POLYGLYCERINE-3 | 2.000 |
| STEARETH-21 | 1.000 |
| POLYMETHYL METHACRYLATE | 1.000 |
| CARBOMER | 0.200 |
| TOCOPHERYL ACETATE | 0.200 |
| PHALAENOPSIS AMABILIS EXTRACT | 0.002 |
| DENDROBIUM FIMBRIATUM EXTRACT | 0.500 |

In addition to its pleasant texture on application, this composition has a remarkable anti-ageing effectiveness, in particular for preventing loss of firmness and elasticity of the skin of the face and of the neck.

5.2. Composition in the Form of a Lotion

TABLE 7

| Ingredients | % by weight |
|---|---|
| AQUA (WATER) | QSP100 |
| GLYCERIN | 3.0000 |
| PENTYLENE GLYCOL | 2.0000 |
| PEG-8 | 2.0000 |
| PEG-7 GLYCERYL COCOATE | 1.0000 |
| PHENOXYETHANOL | 0.8903 |
| PEG-60 HYDROGENATED CASTOR OIL | 0.5000 |
| SODIUM CITRATE | 0.0850 |
| SODIUM HYALURONATE | 0.0400 |
| CITRIC ASSET | 0.0138 |
| PEG-40 HYDROGENATED CASTOR OIL | 0.0090 |
| PHALAENOPSIS AMABILIS EXTRACT | 0.0003 |
| DENDROBIUM FIMBRIATUM EXTRACT | 0.4000 |

Applied on the skin, in particular on the face, this composition provides a firming and smoothing anti-ageing action.

### REFERENCES

Loewus, Improvement in Anthrone Method for Determination of Carbohydrates; Analytical Chemistry, 24(1), 219-219—January 1952.
Ainsworth, E., Gillespie, K. Estimation of total phenolic content and other oxidation substrates in plant tissues using Folin-Ciocalteu reagent. Nat Protoc 2, 875-877 (2007).

1. A no-rinse cosmetic composition for topical application on keratin materials, comprising an extract of Dendrobium fimbriatum.

2. The cosmetic composition according to claim 1, wherein the extract of Dendrobium fimbriatum is present with a content ranging from 0.01% to 10% by weight of raw material, relative to the total weight of the composition.

3. The cosmetic composition according to claim 1 further comprising an extract from orchid root of the species Gastrodia elata.

4. The cosmetic composition according to claim 1 further comprising at least one cosmetic adjuvant chosen from the group consisting of antioxidants, emollients, moisturisers, anti-ageing agents, perfumes, and the mixtures thereof.

5. (canceled)

6. A cosmetic method for care and/or make-up of keratin materials, comprising the topical application on said keratin materials of a cosmetic composition as defined in claim 1.

7. The cosmetic method according to claim 6, for preventing and/or reducing the signs of skin ageing.

8. The cosmetic method according to claim 7, wherein said cosmetic composition is applied on skin that is aged or showing signs of ageing.

9. An extract of Dendrobium fimbriatum, wherein it is obtained by an extraction method comprising at least the steps of:
a) grinding the plant material;
b) at least one extraction step followed by a filtration step;
c) concentrating the extract obtained at the end of step b);
d) adding butylene glycol, pentylene glycol, potassium sorbate/sodium benzoate, glycerol or propanediol;
e) filtering, and
f) obtaining an extract of Dendrobium fimbriatum.

10. The extract of Dendrobium fimbriatum according to claim 9, wherein it is obtained from the entire plant.

11. The extract of Dendrobium fimbriatum according to claim 9, wherein it comprises at least one metabolite chosen from betaine, cis melilotoside, and trans melilotoside.

12. A method for obtaining an extract of Dendrobium fimbriatum comprising at least the steps of:
a) grinding the plant material;
b) at least one extraction step followed by a filtration step;
c) concentrating the extract obtained at the end of step b);
d) adding butylene glycol, pentylene glycol, potassium sorbate/sodium benzoate, glycerol or propanediol;
e) filtering, and
f) obtaining an extract of Dendrobium fimbriatum.

13. The cosmetic composition according to claim 1 wherein the extract of Dendrobium fimbriatum is an extract of Dendrobium fimbriatum var. oculatum.

14. The cosmetic method according to claim 6, for preventing and/or reducing loss of firmness, loss of elasticity, reduction in thickness of the skin, and the appearance of wrinkles and/or fine lines.

15. The cosmetic method according to claim 6, wherein the extract of Dendrobium fimbriatum is an extract of Dendrobium fimbriatum var. oculatum.

16. The Dendrobium fimbriatum extract of claim 9, wherein said extract is an extract of Dendrobium fimbriatum var. oculatum.

17. The method for obtaining an extract of Dendrobium fimbriatum according to claim 12, wherein said extract is an extract of Dendrobium fimbriatum var. oculatum.
